(19) United States
(12) Patent Application Publication
(10) Pub. No.: US 2005/0244806 A1
(43) Pub. Date: Nov. 3, 2005

(54) Mutant proteins and use thereof for the manufacture of medicaments and the treatment of humans or animals suffering from conformational diseases

(76) Inventor: Ralph Zahn, Zurich (CH)

Correspondence Address: NATH & ASSOCIATES, 1030 15th STREET, NW, 6TH FLOOR, WASHINGTON, DC, 20005, US

(21) Appl. No.: 10/520,511

(22) PCT Filed: Jul. 5, 2003
(86) PCT No.: PCT/EP03/07224

§ 371 Date: Jan. 7, 2005

Related U.S. Application Data
(60) Provisional application No. 60/395,021, filed on Jul. 11, 2002.

(51) Int. Cl. C12Q 1/00; A61K 38/17; C07K 14/47; C07H 21/04; C12N 15/09

(52) U.S. Cl. 435/4; 435/69.1; 435/320.1; 435/325; 514/12; 530/350; 536/23.2

(57) ABSTRACT

The invention relates to a mutant prion protein (PrP), the globular domain of which comprises an engineered second disulfide bond in a similar position as in the human doppel protein (hDpl). In an embodiment, the prion protein has an engineered extra disulfide bond in the presumed ‘factor X’ binding epitope and is accommodated with slight, strictly localized conformational changes to inhibit prion propagation in human and animals. Also disclosed is the use of a mutant prion protein (PrP), the globular domain of which comprises at least one engineered additional disulfide bond in a similar position as in the human doppel protein, or fragments thereof for therapeutic treatment or for the manufacture of a medicament for therapeutic treatment of proteins causing disease after a conformational transition, e.g. Tranmissible Spongiform Encephalopathy (TSE), variant forms of Creutzfeldt-Jakob disease (CJD), fatal familial insomnia (FFI), and Gerstmann-Sträussler-Scheinker syndrome (GSS) in human. Further, the use of the PrP mutant protein for in vivo generation of disulfide mutants of prion proteins or fragments thereof is carried out in order to enable an intended ther-apy of TSE in animals, e.g. by somatic gene therapy with lentiviral vector, where TSE includes bovine spongiform encephalopathy (BSE), scrapie in sheep, feline spongiform encephalopathy (FSE), and chronic wasting disease (CWD) in elk and deer.

### RELATED APPLICATION DATA

This patent application claims priority of the U.S. provisional application No. 60/395,021 filed on Jul. 11, 2002 the entire disclosure of which is incorporated herein by reference.

### BACKGROUND OF THE INVENTION

Although the central paradigm of protein folding (Anfinsen, C. B. (1973) Principles That Govern Folding of Protein Chains. Science, 181, 223-230), that the unique three-dimensional structure of a protein is encoded in its amino acid sequence, is well established, its generality has been questioned due to the recently developed concept of “prions”. Biochemical characterization of infectious scrapie material causing central nervous system degeneration indicates that the necessary component for disease propagation is proteinaceous (Prusiner, S. B. (1982) Novel proteinaceous infectious particles cause scrapie. Science, 216, 136-144), as first outlined by (Griffith, J. S. (1967) Self-replication and scrapie. Nature, 215, 1043-1044) in general terms. Prion propagation further involves a conversion from a cellular prion protein, denoted PrP^{C}, into a toxic scrapie form, PrP^{Sc}, which is facilitated by PrP^{Sc} acting as a template for PrP^{C} to form new PrP^{Sc} molecules (Prusiner, S. B. (1987) Prions and neurodegenerative diseases. N Engl J Med, 317, 1571-1581). The “protein-only” hypothesis implies that the same polypeptide sequence, in the absence of any post translational modifications, can adopt two considerably different stable protein conformations. Thus, in the case of prions it is possible, although not proven, that they violate the central paradigm of protein folding. There is some indirect evidence that another factor, provisionally named “protein X”, might be involved in the conformational conversion process (Prusiner, S. B. (1998) Prions. Proc Natl Acad Sci USA, 95, 13363-13383), which includes a dramatic change from α-helical into β-sheet secondary structure. Although it has been proposed that “protein X” might act as a molecular chaperone, the chemical nature of this “factor X” has not been identified yet (Zahn, R. (1999) Prion propagation and molecular chaperones. Q Rev Biophys, 32, 309-370).

Two general models have been proposed for the molecular mechanism by which PrP^{Sc} promotes the conversion of the cellular isoform (see FIG. 1). The “nucleated polymerization” or “seeding” model for PrP^{Sc} formation (Jarrett, J. T. and Lansbury, P. T., Jr. (1993) Seeding “one-dimensional crystallization” of amyloid: a pathogenic mechanism in Alzheimer's disease and scrapie? Cell, 73, 1055-1058) proposes that PrP^{C} and PrP^{Sc} are in a rapidly established equilibrium, and that the conformation of PrP^{Sc} is thermodynamically stable only when trapped within a crystal-like seed (see FIG. 1A). The proposed process is akin to other well-characterized nucleation-dependent protein polymerization processes, including microtubule assembly, flagellum assembly, and sickle-cell hemoglobin fibril formation, where the kinetic barrier is imposed by nucleus formation around single molecules. To explain exponential conversion rates, it must be assumed that the aggregates are continuously fragmented to present increasing surface for accretion, although the mechanism of fragmentation remains to be explained. The “template-assisted” or “heterodimer” model for PrP^{Sc} formation (Prusiner, S. B., Scott, M., Foster, D., Pan, K. M., Groth, D., Mirenda, C., Torchia, M., Yang, S. L., Serban, D., Carlson, G. A. and et al. (1990) Transgenetic studies implicate interactions between homologous PrP isoforms in scrapie prion replication. Cell, 63, 673-686) proposes that PrP^{C} is unfolded to some extent and refolded under the influence of a PrP^{Sc} molecule functioning as a template (see FIG. 1B). A high energy barrier is postulated to make this conversion improbable without catalysis by preexisting PrP^{Sc}. The conformational change is proposed to be kinetically controlled by the dissociation of a PrP^{C}-PrP^{Sc} heterodimer into two PrP^{Sc} molecules, and can be treated as an induced fit enzymatic reaction following autocatalytic Michaelis-Menten kinetics. Once conversion has been initiated it gives rise to an exponential conversion cascade as long as the PrP^{Sc} dimer dissociates rapidly into monomers. A disadvantage of the template-assisted model is that it does not explain why PrP^{Sc} after propagation should aggregate into protein fibrils. Manfred Eigen has presented a comparative kinetic analysis of the two proposed mechanisms of prion disease (Eigen, M. (1996) Prionics or the kinetic basis of prion diseases. Biophysical Chemistry, 63, A1-A18). He found that logically both models are possible, in principle, but that the conditions under which they work seem to be too narrow and unrealistic. The autocatalytic template-assisted model requires cooperativity in order to work, but it then becomes phenomenologically indistinguishable from the nucleation model which is also a form of (passive) autocatalysis. Though the two kind of mechanisms still may differ on the question which of the two monomeric protein conformations is the favored equilibrium state, they both require an aggregated state as the from that is eventually favored at equilibrium and that presumably resembles the pathogenic form of the prion protein. Eigen concluded that more experimental evidence is needed in order to judge which of the two models is the right one. In principle, neither of the models for prion propagation does rule out a possible assistance by “factor X”.

A mechanistic understanding of prion diseases requires a detailed knowledge of the three-dimensional structure of both the cellular form and the pathogenic form of the prion protein. Only if both protein structures have been deciphered one can understand how a conversion takes place. In vivo, the “healthy” prion protein is attached to the cell surface via a glycosyl phosphatitylinositol anchor and partitions to membrane domains that have been termed lipid rafts (Vey, M., Pilkuhn, S., Wille, H., Nixon, R., DeArmond, S. J., Smart, E. J., Anderson, R. G., Taraboulos, A. and Prusiner, S. B. (1996) Subcellular colocalization of the cellular and scrapie prion proteins in caveolae-like membranous domains. Proc Natl Acad Sci USA, 93, 14945-14949). Recent structural studies have focused on soluble recombinant prion proteins from various species using nuclear magnetic resonance (NMR) spectroscopy. These studies show that mammalian PrP^{C} consists of two distinct domains: a flexibly disordered N-terminal tail, which comprises residues 23-120, and a well structured C-terminal globular domain of residues 121-230 that is rich in α-helix secondary structure and contains a small anti-parallel β-sheet (Lopez Garcia, F., Zahn, R., Riek, R. and Wüthrich, K. (2000) NMR structure of the bovine prion protein. Proc Natl Acad Sci USA, 97, 8334-8339). Upon conversion of PrP^{C} into PrP^{Sc}, residues 90-120, which represent the most conserved sequence element in mammalian and non-mammalian prion proteins (Wopfner, F., Weidenhofer, G., Schneider, R., von Brunn, A., Gilch, S., Schwarz, T. F., Werner, T. and Schatzl, H. M. (1999) Analysis of 27 mammalian and 9 avian prion proteins reveals high conservation of flexible regions of the prion protein. J Mol Biol, 289, 1163-1178), become resistant to treatment with proteinase K (Prusiner, S. B., Groth, D. F., Bolton, D. C., Kent, S. B. and Hood, L. E. (1984) Purification and structural studies of a major scrapie prion protein. Cell, 38, 127-134), implying that this polypeptide segment becomes structured. There is further evidence that the conformational transition of PrP^{C} is accompanied by a substantial increase of the β-sheet secondary structure (Pan, K. M., Baldwin, M., Nguyen, J., Gasset, M., Serban, A., Groth, D., Mehlhorn, I., Huang, Z., Fletterick, R. J., Cohen, F. E. and et al. (1993) Conversion of alpha-helices into beta-sheets features in the formation of the scrapie prion proteins. Proc Natl Acad Sci USA, 90, 10962-10966).

### PROBLEMS OBSERVED IN PRION ART

It has been known since the structure determination of mPrP(121-231) that the molecular surface of the potential protein X epitope is formed by two polypeptide segments with low homology among different species, which are close in the three-dimensional structure (Billeter, M., Riek, R., Wider, G., Hornemann, S., Glockshuber, R. and Wüthrich, K. (1997) Prion protein NMR structure and species barrier for prion diseases. Proc. Natl. Acad. Sci USA 94, 7281-7285). The two segments of helix 3 and the loop 165-172 (see FIG. 2) are characterized by significant alterations of the electrostatic surface potential among different mammalian species: human PrP differs from bovine and mouse PrP in the replacement of the glutamine residues 168 and 219 by glutamic acid residues, as well as by conservative substitutions at positions 166, 215, and 220. Studies with scrapie-infected neuroblastoma cells transfected with a chimeric human/mouse Prnp confirmed that single amino acid replacements in the area of the protein X epitope affect the efficiency with which recombinant PrP is converted into PrP^{Sc} (Kaneko, K., Zulianello, L., Scott, M., Cooper, C. M., Wallace, A. C., James, T. L., Cohen, F. E. and Prusiner, S. B. (1997). Evidence for protein X binding to a discontinuous epitope on the cellular prion protein during scrapie prion propagation. Proc. Natl. Acad. Sci. USA 94, 10069-10074). Substitution of residues 168, 215 or 219 with the corresponding residues in human PrP, but not at position 220, diminishes or prevents conversion to PrP^{Sc} in the recombinant construct. The relative affinities of mutant and wild-type protein for protein X were determined by co-transfection studies of mutant and wild-type PrP, which showed that substitution of a basic residue for glutamines occurring at positions 168, 172, or 219 inhibits conversion of both mutant and wild-type PrP owing to the failure of the mutated PrP^{C} to release protein X. Conversely, exchange of glutamines 168 or 219 against glutamic acid prevented conversion of mutant PrP but allowed conversion of wild-type PrP, presumably by weakening mutant PrP^{C}-protein X binding.

These studies show that in cell culture single amino acid substitutions within the presumed factor X epitope of a mutant prion protein are able to suppress the wild-type PrP^{C} to PrP^{Sc} conversion. Somatic gene therapy thus appears to be a promising strategy for treatment of Transmissible Spongiform Encephalopathy (TSE) in human and animals. However, it remains to be established whether substitutions of this kind also have the capacity to inhibit prion propagation in human and animals over a sufficient period of time, without affecting the physiological function of wild-type PrP^{C}, which in fact might be dependent of a functional interaction with protein X.

### OBJECT AND SUMMARY OF THE INVENTION

It is therefore an object of the present invention to provide PrP proteins which inhibit prion propagation in human and animals over a sufficient period of time. This object is attained by the features of claim 1.

An other object of the present invention is to provide a use of the PrP proteins or fragments thereof for therapeutic treatment or for the manufacture of a medicament as well as a medicament for therapeutic treatment of proteins causing disease after a conformational transition.

Advantageous embodiments and additional characteristics in accordance with the invention ensue from the dependent claims.

This invention describes the nuclear magnetic resonance (NMR) structure of the globular domain with residues 121-230 of a mutant human prion protein with two disulfide bonds, hPrP(M166C/E221C), containing a second disulfide bond in a similar position as in the human doppel protein (hDpl). Another mutant, hPrP(M166C/Y225C), was expressed and shown to fold into a globular structure, but its tendency to aggregate precluded a detailed structural analysis. The NMR structure hPrP(M166C/E221C) shows the same global fold as wild-type hPrP(121-230). It contains three α-helices of residues 144-154, 173-194 and 200-228, an anti-parallel β-sheet of residues 128-131 and 161-164, and the disulfides Cys166-Cys221 and Cys179-Cys214. The engineered extra disulfide bond in the presumed ‘factor X’ binding site is accommodated with slight, strictly localized conformational changes. High compatibility of hPrP with insertion of a second disulfide bridge in the factor X epitope was further substantiated by model calculations with additional variant structures. The ease with which the hPrP structure can accommodate a variety of locations for a second disulfide bond within the presumed factor X binding epitope strongly suggests a functional role for the observed extensive perturbation of the corresponding region in hDpl by the natural second disulfide bond. The functional role of the second disulfide bond in Dpl, and possibly also in the mutant prion proteins, might include the propensity to resist a conformational transition into a pathogenic isoform causing Transmissible Spongiform Encephalopathy (TSE) such as Creutzfeldt-Jakob disease (CJD) in human.

### BRIEF DESCRIPTION OF THE FIGURES

The following figures are intended to document prior art as well as the invention. Preferred embodiments of the method in accordance with the invention will also be explained by means of the figures, without this being intended to limit the scope of the invention.

FIG. 1. Two general models proposed for the molecular mechanism by which PrP^{Sc} promotes the conversion of the cellular isoform (Zahn, R. (1999):

FIG. 1A The “nucleated polymerization” or “seeding” model;

FIG. 1B The “template-assisted” or “heterodimer” model;

FIG. 2. Amino acid sequence alignment of the human prion protein segment 165-230 and the human doppel protein segment 93-153;

FIG. 3. Two-dimensional [¹⁵N,¹H]-correlation spectroscopy (COSY) spectra of (A) hPrP(M166C/E221C) and (B) hPrP(M166C/Y225C);

FIG. 4. Stereo views of the NMR structure of hPrP(M166C/E221C):

FIG. 4A Backbone of 20 energy-refined DYANA conformers superimposed for best fit of the N, C° and C′ atoms of residues 125-228;

FIG. 4B All-heavy-atom representation of the conformer from (A);

FIG. 4C Ribbon drawing of the conformer from (B);

FIG. 5. Plots versus the hPrP(121-230) amino acid sequence of ¹³C^{α} chemical shift differences, Δδ(¹³C^{α}):

FIG. 5A hPrP(M166C/E221C) versus the random coil shifts;

FIG. 5B hPrP(M166C/Y225C) versus the random coil shifts;

FIG. 5C hPrP(M166C/E221C) versus wild-type hPrP(121-230);

FIG. 5D hPrP(M166C/Y225C) versus wild-type hPrP(121-230);

FIG. 6. Steady-state ¹⁵N{¹H}-NOEs of hPrP(M166C/E221C) and hPrP(121-230);

FIG. 7 GdmCl-dependent mean residue molar ellipticity of human prion proteins:

FIG. 7A In buffer containing 20 mM sodium phosphate at pH 7.0;

FIG. 7B In buffer containing 20 mM sodium acetate at pH 5.0;

FIG. 8 Circular dichroism spectra of human prion proteins:

FIG. 7A hPrP(121-230);

FIG. 7B hPrP(M166C/E221C);

FIG. 7C hPrP(M166C/Y225C);

FIG. 9 Temperature-dependent mean residue molar ellipticity of human prion proteins:

FIG. 9A hPrP(121-230);

FIG. 9B hPrP(M166C/Y225C);

FIG. 9C hPrP(M166C/E221C).

### DETAILED DESCRIPTION OF THE INVENTION

The three-dimensional structures of the human prion protein and the human doppel protein show a similar folding topology (Thorsten Lührs, Roland Riek, Peter Güntert und Kurt Wüthrich, submitted; Zahn et al., 2000), with a flexibly disordered N-terminal ‘tail’ attached to a 100-residue globular C-terminal domain containing three α-helices and a small anti-parallel β-sheet. A striking difference between these two proteins concerns the number of disulfide bonds. In both hPrP and hDpl, a disulfide bridge linking the helices α2 and α3 is buried within the hydrophobic core, and contributes significantly to overall stability of the globular protein structure. It has been shown that reduction of the Cys residues 179 and 214 with dithiothreitol results in unfolding and aggregation of PrP in vitro (Mehlhorn, I., Groth, D., Stöckel, J., Moffat, B., Reilly, D., Yansura, D., Willett, W. S., Baldwin, M., Fletterick, R., Cohen, F. E., Vandien, R., Henner, D. and Prusiner, S. B. (1996) High-level expression and characterization of a purified 142-residue polypeptide of the prion protein. Biochemistry 35, 5528-5537), implying that the so far unknown physiological functions of PrP, and presumably also Dpl are dependent on this intact disulfide bond.

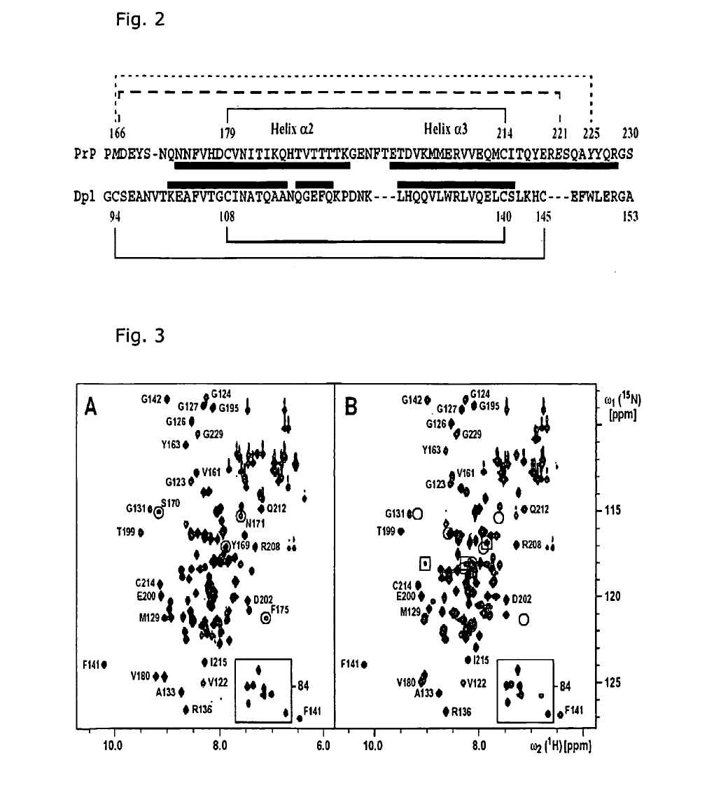

In Dpl, the loop between α-strand 2 and helix α2 is connected to a sequence position near the C-terminus by an additional disulfide bond, which has no counterpart in wild-type PrP (FIG. 2).

FIG. 2 shows amino acid sequence alignment of the human prion protein segment 165-230 and the human doppel protein segment 93-153 based on consideration of the sequences as well as the three-dimensional structures of the two proteins (T. Lührs, R. Riek, P. Güntert and K. Wüthrich, submitted; Zahn, R., Liu, A., Lührs, T., Riek, R., von Schroetter, C., López Garcia, F., Billeter, M., Calzolai, L., Wider, G. and Wüthrich, K. (2000) NMR solution structure of the human prion protein. Proc. Natl. Acad. Sci. USA 97, 145-150). The residue positions in hPrP that were exchanged against Cys in this paper are in italics. Solid black lines indicate the natural disulfide bridges, and the locations of the regular α-helical secondary structures in the wild-type proteins are indicated by black boxes. The broken and dotted lines indicate, respectively, the extra disulfide bond in hPrP(M166C/E221C), for which a complete structure was obtained, and in hPrP(M166C/Y225C), which was also expressed and characterized.

The corresponding region of PrP is devoid of cysteinyl residues and has been suggested, on the basis of inoculation studies with transgenic mice expressing various PrP constructs (Telling, G. C., Scott, M., Mastrianni, J., Gabizon, R., Torchia, M., Cohen F. E., DeArmond, S. J. and Prusiner, S. B. (1995) Prion propagation in mice expressing human and chimeric PrP transgenes implicates the interaction of cellular PrP with another protein. Cell 83, 79-90), to represent an epitope for binding of a not yet further identified ‘factor X’ that supposedly participates in the disease-related conformational transformation of PrP in vivo (Prusiner, 1998).

The NMR structures of the human and murine Dpl proteins show that the introduction of a second disulfide bond in the region corresponding to the presumed ‘factor X’ binding epitope in PrP results in a mayor change of the three-dimensional structure. To investigate the effect of an artificial extra disulfide bond in this region of the three-dimensional prion protein structure we generated two mutants of the globular domain of the human prion protein, hPrP(121-230). hPrP(M166C/E221C) and hPrP(M166C/Y225C) were designed so as to simultaneously mimic the location of the second disulfide bond in hDpl (FIG. 2) and to be compatible with the three-dimensional structure of wild-type human PrP (Zahn et al., 2000). Among the sites thus chosen for the amino acid substitutions in hPrP, Glu221 is fully conserved in the amino acid sequences of 27 mammalian and 9 avian prion proteins (Wopfner et al., 1999), Tyr225 is replaced with Ser, Phe or Ala in some of the species, and Val166 is replaced by Met or Ile only in human, chimpanzee and marsupial PrP. Among the known Dpl sequences (Moore, R. C., Lee, I. Y., Silverman, G. L., Harrison, P. M., Strome, R., Heinrich, C., Karunaratne, A., Pasternak, S. H., Chishti, M. A., Liang, Y., Mastrangelo, P., Wang, K., Smit, A. F. A., Katamine, S., Carlson, G. A., Cohen, F. E., Prusiner, S. B., Melton, D. W., Tremblay, P., Hood, L. E. and Westaway, D. (1999) Ataxia in prion protein (PrP)-deficient mice is associated with upregulation of the novel PrP-like protein doppel. J. Mol. Biol. 292, 797-817), the residues Cys94 and Cys145 in the second disulfide bond are fully conserved.

This invention describes a high-quality NMR structure of hPrP(M166C/E221C), a qualitative spectroscopic characterization of hPrP(M166C/Y225C), and model calculations of additional two-disulfide mutants of hPrP(121-230). The results are evaluated with regard to possible functional roles of the factor X binding epitope in PrP and the corresponding molecular region in Dpl.

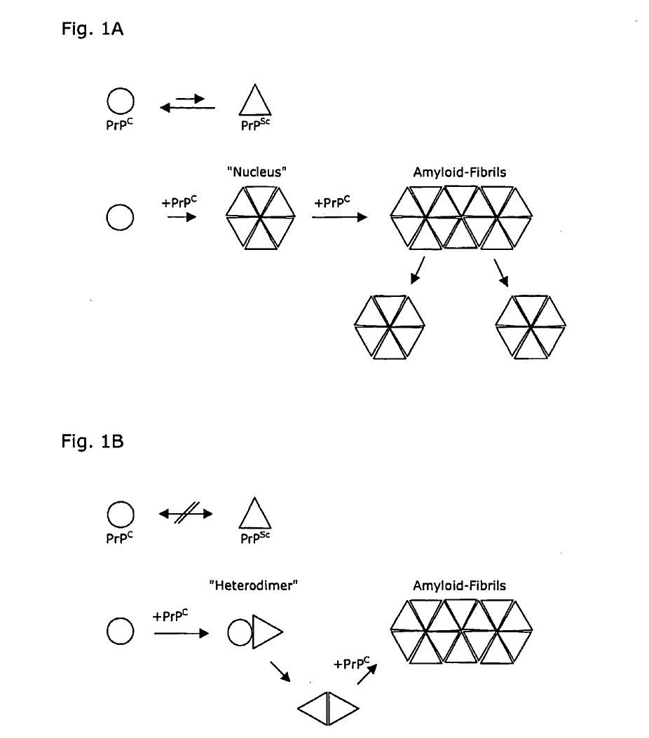

Furthermore, this invention includes the following applications:
1) The generation of ‘disulfide mutants’ of prion proteins or fragments thereof for therapeutic treatment of Transmissible Spongiform Encephalopathy (TSE), in particular of mutant proteins where additional disulfide bonds are introduced between segment 165-175 and the C-terminal residues 215-230. The additional disulfide bond(s) might prevent a conformational transition of mutant PrP^{C} into PrP^{Sc}, and thus suppress a conformational transition of PrP^{C} into PrP^{Sc} in co-existing wild-type protein by dominant negative inhibition of the following kind:
   a) Binding of mutant PrP^{C} to wild-type PrP^{C}, thus suppressing a conformational transition into wild-type PrP^{Sc} oligomers (see FIG. 1A).
   b) Binding of mutant PrP^{C} to wild-type PrP^{Sc} oligomers, thus suppressing the formation of wild-type PrP^{Sc} amyloid fibrils (see FIG. 1A).
   c) Binding of mutant PrP^{C} to wild-type PrP^{Sc}, thus suppressing the formation of wild-type PrP^{C}/PrP^{Sc} heterodimers (see FIG. 1B).
   d) Binding of mutant PrP^{C} to wild-type PrP^{Sc} amyloid fibrils, thus suppressing the elongation of amyloid fibrils (see FIG. 1A,B) or the dissociation of amyloid fibrils into PrP^{Sc} oligomers (see FIG. 1A).
2) The in vivo generation of disulfide mutants of prion proteins or fragments thereof in humans or cell cultures for therapy of TSE, e.g. by somatic gene therapy with vectors, like the lentiviral vector, plasmids or liposomes, where TSE includes spontaneous, inherited, iatrogenic and variant forms of Creutzfeldt-Jakob disease (CJD), fatal familial insomnia (FFI), and Gerstmann-Sträussler-Scheinker syndrome (GSS).
3) The recombinant production of disulfide mutants of prion proteins or fragments thereof for therapy of TSE in human, e.g. by direct application of the recombinant protein, where TSE includes spontaneous, inherited, iatrogenic and variant forms of CJD, FFI, and GSS.
4) The in vivo generation of disulfide mutants of prion proteins or fragments thereof in animals or cell cultures for therapy of TSE, e.g. by somatic gene therapy with vectors, like the lentivfral vector, plasmids or liposomes, where TSE includes bovine spongiform encephalopathy (BSE), scrapie in sheep, feline spongiform encephalopathy (FSE), and chronic wasting disease (CWD) in elk and deer.
5) The recombinant production of disulfide mutants of prion proteins or fragments thereof for therapy of TSE in animals, e.g. by direct application of the recombinant protein, where TSE includes BSE, scrapie, FSE and CWD.
6) The recombinant production of disulfide mutants of prion proteins or fragments thereof as “conversion-resistant PrP^{C} standard” for TSE-tests, where recombinant PrP^{C} is amplified by PrP^{Sc} from pathogenic tissue or bodily fluid such as blood and urine. TSE-tests may be applied to human and animals such as cattle, cat, sheep, elk, deer, pig, horse, and chicken.
7) The in vivo generation of disulfide mutants of prion proteins or fragments thereof for breeding of TSE-resistant animals, where animals include cattle, sheep, cat, elk and deer.
8) The invention and its applications may be applied to other proteins involved in neurodegenerative diseases (e.g. Alzheimers, Parkinsons disease, Multiple sclerosis) or generally to proteins causing disease after a conformational transition (conformational diseases such as Primary systematic amyloidosis, Type II diabetes, Atrial amyloidosis).

The invention further includes generation and/or application of wild type proteins according to this points 1-8 or variants thereof. Such variants comprise protein fragments, mutant proteins, fusion proteins, and protein-ligand complexes.

### EXPERIMENTAL RESULTS

1. Production and Spectroscopic Characterization of Two Mutant Prion Proteins:

The two mutant proteins hPrP(M166C/E221C) and hPrP(M166C/Y225C) were expressed as inclusion bodies in E. coli and purified by high-affinity column refolding, which resulted in similar yield as for wild-type hPrP(121-230) (Zahn, R., von Schroetter, C. and Wüthrich, K. (1997) Human prion proteins expressed in Escherichia coli and purified by high-affinity column refolding. FEBS Lett. 417, 400-404; Zahn et al., 2000). The formation of an additional disulfide bond was confirmed by mass spectrometry, and resulted in an increase of the melting temperature by about 10° C. for both proteins (data not shown). The mutant proteins were uniformly ¹³C,¹⁵N-labeled for resonance assignment and structure determination. Solutions containing 1 mM protein and 10 mM sodium acetate at pH 4.5 and 20° C. were used for the NMR experiments.

The ¹H NMR spectra of both proteins showed that the preparations are homogeneous, and the chemical shift dispersion is typical for globular proteins. Closely similar structures are clearly apparent by the chemical shifts observed by two-dimensional [¹⁵N,¹H]-correlation spectroscopy (COSY) (FIG. 3).

FIG. 3 shows two-dimensional [¹⁵N,¹H]-COSY spectra of (A) hPrP(M166C/E221C) and (B) hPrP(M166C/Y225C). Selected cross peak assignments are indicated with black lettering. In FIG. 3A, circles and lettering indicate cross peaks that have been observed for hPrP(M166C/E221C), but have not been seen either in the spectra of hPrP(M166C/Y225C) or wild-type hPrP(121-230) (Zahn et al., 2000). In FIG. 3B, empty circles identify positions where cross peaks were expected from comparison with (A) and with hPrP(121-230), and rectangles indicate cross speaks that could not be assigned because of missing sequential connectivities in the triple-resonance spectra. In both spectra the rectangular frame encloses folded cross peaks of H^{Nε} in Arg side chains. The spectra were recorded at 600 MHz with 1 mM protein solutions in 90% H₂O/10% D₂O, 10 mM [d₄]-sodium acetate at pH 4.5 and T=20° C.

However, whereas in the spectrum of hPrP(M166C/E221C) (FIG. 3A) all 108 expected backbone amide resonances were observed (the thrombin cleavage site adds a Gly-Ser dipeptide preceding the N-terminus of the prion protein sequence (Zahn et al., 1997), so that the resonances of Ser120 and Val121 are also observed in the [¹⁵N,¹H]-COSY spectrum), we could identify only 103 backbone amide resonances in hPrP(M166C/Y225C) (FIG. 3B). Throughout, the resonance lines of the amide protons in hPrP(M166C/Y225C) are broadened by about 6 Hz in comparison with spectrum A, which indicates transient aggregation of this mutant protein into oligomers.

2. Resonance Assignment and Structure Determination of hPrP(M166C/E221C):

Sequence-specific backbone assignments were obtained using standard triple-resonance experiments with the ¹³C,¹⁵N-labeled protein (Bax, A. and Grzesiek, S. (1993) Methodological advances in protein NMR. Acc. Chem. Res. 26, 131-138), and the sequence-specific assignments were independently confirmed by sequential and medium-range nuclear Overhauser enhancement (NOE) cross peaks (Wüthrich, K. (1986) NMR of Proteins and Nucleic Acids, Wiley, N.Y.). All polypeptide backbone resonances were assigned, including the amide nitrogens and amide protons of all the residues in the loop 165-175 and of Phe175 (FIG. 3A), which were not detected in the wild-type protein (Zahn et al., 2000). At least either one heteronuclear sequential scalar connectivity or a sequential NOE has been observed for each pair of neighboring residues. The side chains were assigned based on chemical shift comparison with wild-type hPrP(121-230) (Zahn et al., 2000), and have been confirmed using a three-dimensional ¹⁵N-resolved [¹H,¹H]-total correlation spectroscopy (TOCSY) spectrum (Marion, D., Kay, L. E., Sparks, S. E., Torchia, D. A. and Bax, A. (1989) Three-dimensional heteronuclear NMR of ¹⁵N-labelled proteins. J. Am. Chem. Soc. 111, 1515-1517). The side chain assignments of non-labile protons are complete, with the sole exceptions of εCH of His155 and His187, and ζCH of Phe175 and Phe198. Among the labile side chain protons, the amide groups of all 7 Asn and Gln residues and the ε-proton resonances of the 8 Arg residues were assigned by intraresidual NOEs (Wüthrich, 1986). Of the side chain hydroxyl protons of Ser, Thr and Tyr only the resonance of Thr183 could be observed and assigned.

The methyl groups of the 9 Val and 2 Leu in hPrP(M166C/E221C) were stereo-specifically assigned, and additional stereospecific assignments were obtained for 2 αCH₂, 30 βOCH₂, 17 γCH₂ and 4 δCH₂ groups, using the program FOUND (Güntert, P., Billeter, M., Ohlenschlager, O., Brown, L. R. and Wüthrich, K. (1998) Conformational analysis of protein and nucleic acid fragments with the new grid search algorithm FOUND. J. Biomol. NMR 12, 543-548) implemented in the DYANA package (Güntert, P., Mumenthaler, C. and Wüthrich, K. (1997) Torsion angle dynamics for NMR structure calculation with the new program DYANA. J. Mol. Biol. 273, 283-298). The ¹³C^{α} resonances of the four Cys residues are all strongly downfield-shifted in the range 39.6 ppm to 41.6 ppm, confirming that they all form disulfide bonds (Wishart et al., 1995). The ¹H, ¹³C and ¹⁵N chemical shifts for hPrP(M166C/E221C) have been deposited with the Biological Magnetic Resonance Bank, file 5378.

A total of 4477 nuclear Overhauser enhancement spectroscopy (NOESY) cross peaks were interactively picked and integrated in a 750 MHz three-dimensional combined ¹⁵N/¹³C-resolved [¹H,¹H]-NOESY recorded in H₂O with a mixing time of 40 ms. Using these peak lists and the chemical shift list as input for the programs CANDID for automated NOE assignment (Herrmann, T., Güntert, P. and Wüthrich, K. (2002) Protein NMR structure determination with automated NOE assignment using the new software CANDID and the torsion angle dynamics algorithm DYANA. J. Mol. Biol. 319, 209-227) and DYANA for structure calculation (Güntert et al., 1997) (see Materials and Methods for details), 1775 NOE upper limit distance constraints were obtained. As supplementary conformational constraints, all residues with ¹³C^{α} chemical shifts deviating from the random coil values by more than 1.5 ppm were subjected to the following bounds on the dihedral torsion angles: −120°<φ<−20° and −100°<ψ<0° for deviations >1.5 ppm; −200°<φ<−80° and 40°<ψ<220° for deviations <−1.5 ppm (Luginbühl, P., Szyperski, T. and Wüthrich, K. (1995) Statistical basis for the use of ¹³C^{α} chemical shifts in protein structure determination. J. Magn. Reson. B 109, 229-233). The combined information from the intraresidual and sequential NOEs, and the ¹³C^{α} chemical shifts used as input for the program FOUND yielded 458 constraints on dihedral angles φ, ψ, X¹ and X². Three upper and three lower distance limits were used to enforce each of the two disulfide bonds Cys166-Cys221 and Cys179-Cys214 (Williamson, M. P., Havel, T. F. and Wüthrich, K. (1985) Solution conformation of proteinase inhibitor IIA from bull seminal plasma by ¹H nuclear magnetic resonance and distance geometry. J. Mol. Biol. 182, 295-315). The final DYANA calculation in the seventh cycle of the CANDID standard protocol (Herrmann et al., 2002) was performed with 100 randomized starting structures, and the 20 best DYANA conformers were further energy-refined with the program OPALp. The resulting bundle of 20 energy-minimized conformers is used to represent the NMR structure. Table I gives a survey of the results of the structure calculation.

TABLE I
Characterization of the 20 energy-refined DYANA conformers
representing the NMR structure of hPrP(M166C/E221C)^{a}:

| | Quantity | Value^{b} |
|---|---|---|
| Residual DYANA target | | 1.05 ± 0.13 |
| function value (Å²)^{c} | | |
| Residual NOE distance | Number > 0.1 Å | 32 ± 5 |
| constraint violations | | |
| | Maximum (Å) | 0.14 ± 0.01 |
| Residual dihedral angle | Number > 2.5 degrees | 0 ± 0 |
| constraint violations | | |
| | Maximum (degrees) | 1.64 ± 0.41 |
| Residual disulfide bond | Number > 0.1 Å | 0 ± 0 |
| constraint violations | | |
| | Maximum (Å) | 0.02 ± 0.02 |
| AMBER energy (kcal/mol) | Total | −4641 ± 92 |
| | van der Waals | −293 ± 14 |
| | Electrostatic | −5285 ± 78 |
| RMSD from ideal geometry | Bond lengths (Å) | 0.0078 ± 0.0002 |
| | Bond angles (degrees) | 1.91 ± 0.04 |
| RMSD, N, C^{α}, C′ (125-228) | | 0.63 ± 0.13 |
| (Å)^{d} | | |
| RMSD, all heavy atoms | | 1.11 ± 0.11 |
| (125-228) (Å)^{d} | | |

^{a}The input consisted of 1775 NOE upper distance constraints, 116 dihedral angle constraints on Φ and ψ, and 6 upper distance and 6 lower distance constraints to enforce the disulfide bonds 166-221 and 179-214.
^{b}Average values ± standard deviations for the 20 energy-minimized conformers with the lowest DYANA target function values are given.
^{c}Before energy minimization.
^{d}RMSD values relative to the mean coordinates.

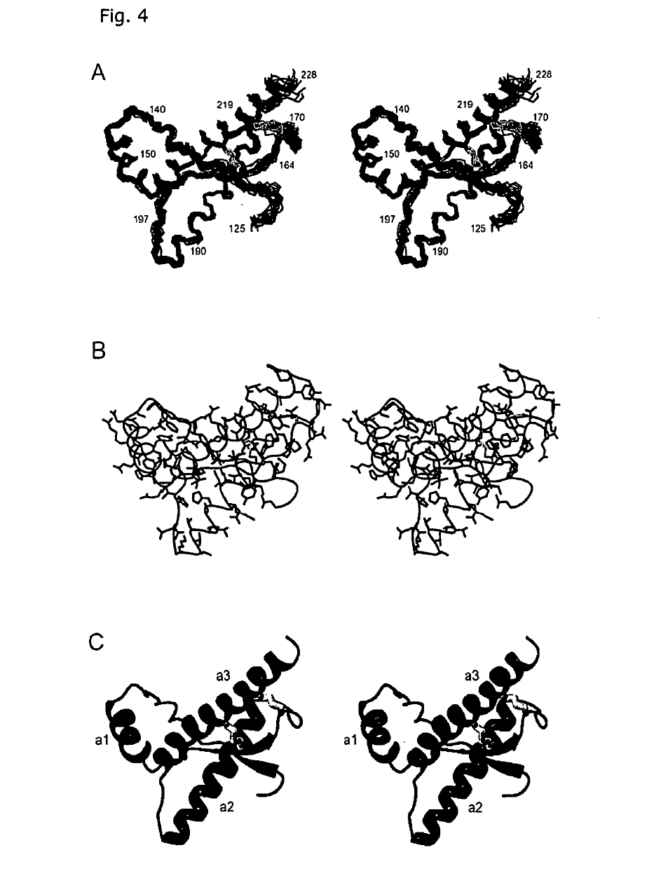

The small residual constraint violations show that the structure is consistent with the experimental constraints, and the global RMSD values among the bundle of 20 conformers is representative of a high-quality structure determination (FIG. 4).

FIG. 4 shows stereo views of the NMR structure of hPrP(M166C/E221C). In FIG. 4A, the backbones of 20 energy-refined DYANA conformers are superimposed for best fit of the N, C^{α} and C′ atoms of residues 125-228. FIG. 4B is an all-heavy-atom representation of the conformer from (A) with the smallest deviation from the mean coordinates, where the backbone is shown as a spline function through the C^{α} positions. FIG. 4C is a ribbon drawing of the conformer from (B). In all drawings the two disulfide bridges are drawn in white.

The atomic coordinates of the bundle of 20 conformers have been deposited with the Protein Data Bank, accession code 1H0L.

3. New Calculation of the Wild-type hPrP(121-230) NMR Structure with the Programs DYANA and CANDID:

Given that the main interest of this work is focused on comparison of mutant human prion proteins with wild-type hPrP(121-230), we re-evaluated the NOE input data and repeated the structure calculation of the previously published hPrP(121-230) structure (Zahn et al., 2000) with the new CANDID/DYANA protocol (Herrmann et al., 2002). This ensures that the structure comparisons in this paper are not influenced by systematic differences that might arise from the use of somewhat different protocols for data analysis and calculation of the new structures of mutant proteins and the reference wild-type structure.

4. Influence of the Additional Disulfide Bond on Conformational Equilibria in the NMR Structure of hPrP(M166C/E221C):

The NMR structure of hPrP(M166C/E221C) has the same global fold as hPrP(121-230), with an RMSD value of 1.08 Å between the backbone heavy atoms of residues 125-228 in the mean structures of the two proteins. Regular secondary structures include a short two-stranded anti-parallel β-sheet with residues 128-131 and 161-164, helix α1 with residues 144-154, helix α2 with residues 173-31 and 161-164, helix α1 with residues 144-154, helix α2 with residues 173-194, and helix α3 with residues 200-228.

Within the framework of the preserved global structure, there are variations in the precision of the structure determination for loop 165-172 in the mutant and wild-type hPrP(121-230). In the wild-type protein, the backbone amide resonances of three amino acids in the loop 165-172 are not observable, presumably because of line broadening attributable to slow conformational exchange on the NMR chemical shift time scale (Zahn et al., 2000). This results in reduced precision of the structure determination for the segment 165-172, because of scarcity of NOE upper distance limit constraints. In contrast, complete polypeptide of backbone assignments were obtained for hPrP(M166C/E221C), which enabled the identification of additional medium-range NOEs in the loop 165-172, which is therefore significantly better defined than in the wild-type protein (FIG. 4A). The disulfide bond between Cys166 and Cys221 thus seems to reduce the conformation space accessible to the loop 165-172 and thus to largely suppress the previously observed exchange broadening of backbone amide resonances in this polypeptide segment (Zahn et al., 2000).

The helix α3 is equally well defined in hPrP(M166C/E221C) and hPrP(121-230), and differences between the two structures are within the conformation space spanned by the bundles of 20 conformers. These observations in the calculated structures correlate with near-identical density of NOE distance constraints, in particular medium-range constraints d_{αN}(i, i+3), d_{αN}(i, i+4) and d_{αβ}(i, i+3), which have a dominant influence on the regular α-helix fold (Wüthrich, 1986). Because of the dependence of the NOE intensity on the inverse sixth power of the distance d, only folded forms of a polypeptide with short d values contribute significantly to the observed NOEs, so that in the presence of rapid conformational equilibria with unfolded forms only the folded structure is usually obtained in a NOE-based NMR structure determination. Different averaging applies to the differences between observed and random coil ¹³C^{α} chemical shifts, Δδ(¹³C^{α}), which can therefore be qualitatively related to the populations of regular secondary structures (Luginbühl et al., 1995; Wishart, D. S. and Sykes, B. D. (1994) The ¹³C Chemical-Shift Index: a simple method for the identification of protein secondary structure using ¹³C chemical-shift data. J. Biomol. NMR 4, 171-180).

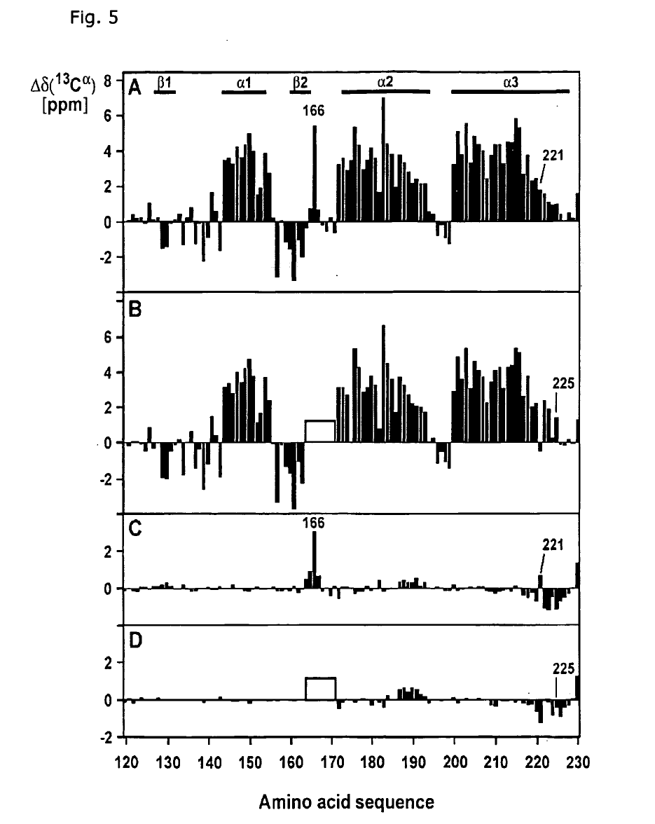

FIG. 5 shows plots versus the hPrP(121-230) amino acid sequence of ¹³C^{α} chemical shift differences, Δδ(¹³C^{α}). (A) and (B), hPrP(M166C/E221C) and hPrP(M166C/Y225C), respectively, versus the random coil shifts (Wishart, D. S., Bigam, C. G., Holm, A., Hodges, R. S. and Sykes, B. D. (1995) ¹H, ¹³C and ¹⁵N random coil NMR chemical shifts of the common amino acids. I. Investigations of nearest-neighbor effects. J. Biomol. NMR 5, 67-81); (C) and (D), hPrP(M166C/E221C) and hPrP(M166C/Y225C), respectively, versus wild-type hPrP(121-230) (Zahn et al., 2000). The positions of the amino acid replacements in the two mutants of hPrP(121-230) are indicated by the sequence positions. The rectangle in (B) and (D) indicates the residues 164-171, for which the ¹³C^{α} chemical shifts could not be assigned in hPrP(M166C/Y225C). In (C) and (D), no data are given for residues 169 and 175, since these ¹³C^{α} chemical shifts could not be assigned in hPrP(121-230). The locations of the regular secondary structure elements are given at the top.

The FIG. 5A shows that although for all ¹³C^{α} atoms located within helix α3 of hPrP(M166C/E221C) the resonances are shifted downfield relative to the random coil shifts, the smaller values of Δδ(¹³C^{α}) for all residues in the C-terminal two turns indicate lower population of the α-helical structure towards the C-terminus. Considering that it appears not to be manifested in the NOE-based structure, this equilibrium seems to be with unfolded forms of the polypeptide. Comparing the values of Δδ(¹³C^{α}) between hPrP(M166C/E221C) and hPrP(121-230) further shows that all but one of the chemical shifts within the segment 222-228 of the mutant protein are shifted upfield (FIG. 5C). The introduction of the additional disulfide bond thus seems to slightly decrease the population of helical structure in the C-terminal two turns of α3.

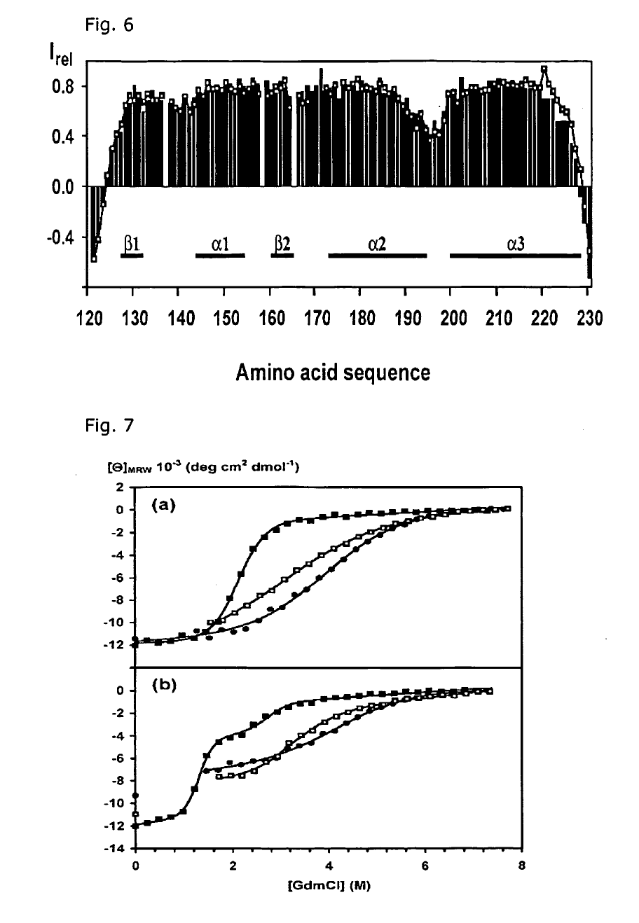

The conformational equilibria manifested in the ¹³C^{α} chemical shifts correlate with intramolecular rate processes that may be detected by measurement of heteronuclear ¹⁵N{¹H}-NOEs. For hPrP(121-230) and hPrP(M166C/E221C), the ¹⁵N{¹H}-NOEs show a uniform distribution over most of the amino acid sequence, with typical values for a globular protein with the size of hPrP(121-230) (FIG. 6).

FIG. 6 shows steady-state ¹⁵N{¹H}-NOEs of hPrP(M166C/E221C) (black bars) and hPrP(121-230) (open squares). For hPrP(121-230), the amide protons of residues 169, 170, 171 and 175 could not be observed because of line broadening. Residues 137, 158 and 165 are prolines. The locations of the regular secondary structure elements are indicated at the bottom.

Besides the last two turns of helix α3, only the residues 121-126, which are unstructured and connect the globular domain with the flexible tail in the intact PrP, and the residues 191-198, which form the somewhat disordered C-terminal end of helix α2 and the subsequent loop (Zahn et al., 2000), show decreased positive or negative ¹⁵N{¹H}-NOE values. In both proteins the helix α3 is thus the only well-defined structural region with somewhat higher-than-average internal mobility, and the introduction of the extra disulfide bond in hPrP(M166C/E221C) causes both a lowering of the population of the α3 helix structure and a slightly increase of its internal mobility.

5. Spectroscopic Characterization of hPrP(M166C/Y225C):

The increased linewidths in the NMR spectra of hPrP(M166C/Y225C) (FIG. 3B) precluded complete backbone assignments. No unambiguous assignments could be obtained for the amide protons and amide nitrogens of Arg164, Cys166, Asp167, Glu168, Tyr169, Ser170, Asn171 and Phe175. In view of both, the limited quality of the NMR data and the results of the model calculations described below, we only completed a ¹³C^{α} chemical shift-based analysis of regular secondary structure. As a result, the ¹³C^{α} chemical shift differences relative to the random coil shifts (FIG. 5B) as well as relative to wild-type hPrP(121-230) (FIG. 5D) show that the secondary structure elements of the wild-type hPrP(121-230) are conserved also for this mutant protein.

6. Model Calculations for Additional hPrP(121-230) mutants with two disulfide bonds:

To investigate the compatibility of the wild-type hPrP(121-230) structure with alternative positioning of an extra disulfide bridge, we used the program DYANA for a series of model calculations. As an input for these calculations we used the same distance and dihedral angle constraints as for the aforementioned new structure determination of hPrP(121-230), except that three upper and three lower distance limits were added to enforce each one of the different individual extra disulfide bonds (Williamson et al., 1985), and all NOE constraints with protons beyond βCH₂ of the residues that were replaced by cysteine were eliminated. The results in Table II show that all the calculations with disulfide constraints linking residues 165 or 166 with either of the residues 221, 222 or 225 converged well, with no or only a slight increase of the residual DYANA target function value when compared to the calculation for hPrP(121-230). Furthermore, the introduction of these disulfide bonds did in no case lead to significant residual upper limit disulfide constraint violations, and the “\overline{RMSD}” of the mutant protein relative to the mean coordinates of hPrP(121-230) was within the conformation space spanned by the 20 conformers. As an internal control we also investigated proteins with disulfide bonds linking one of the wild-type Cys with one of the artificial Cys residues. These calculations did properly converge, but the resulting structures showed dramatically increased target function values, disulfide bond constraint violations, and \overline{RMSD} values.

TABLE II
Characterization of two-disulfide mutants of hPrP(121-230) calculated
after adding constraints for the introduction of an extra disulfide bond
to the input used previously for the structure determination of wild-
type hPrP(121-230)^{a}.

| Disulfide bond constraints | NOEs^{b} | Target Function^{c} | Violations^{d} | RMSD^{e} | RMSD^{f} |
|---|---|---|---|---|---|
| 179-214 (wild-type) | 1798 | 0.74 ± 0.07 | 0 ± 0 | 0.65 ± 0.10 | |
| 166-221, 179-214 | 1767 | 0.73 ± 0.07 | 0 ± 0 | 0.62 ± 0.12 | 0.54 |
| 166-225, 179-214 | 1767 | 0.76 ± 0.08 | 0 ± 0 | 0.72 ± 0.11 | 0.47 |
| 165-221, 179-214 | 1777 | 0.78 ± 0.08 | 0 ± 0 | 0.67 ± 0.13 | 0.26 |
| 165-222, 179-214 | 1780 | 1.09 ± 0.15 | 0 ± 0 | 0.66 ± 0.12 | 0.66 |
| 165-225, 179-214 | 1776 | 0.84 ± 0.10 | 0 ± 0 | 0.71 ± 0.09 | 0.99 |
| 166-222, 179-214 | 1770 | 0.80 ± 0.08 | 0 ± 0 | 0.65 ± 0.16 | 0.42 |
| 166-179, 214-221 | 1767 | 23.1 ± 0.40 | 4 ± 1 | 0.80 ± 0.12 | 3.47 |
| 166-214, 179-221 | 1767 | 14.8 ± 0.45 | 5 ± 0 | 0.83 ± 0.23 | 3.58 |
| ^{a}Eight different mutant proteins were generated by combination of the two natural Cys residues and two artificial extra Cys residues into two disulfide bonds, where the extra cysteines occupy five different sequence positions. The input for the structure calculations was adapted from the input for wild-type hPrP(121-230), the data of which are also listed In the top row for comparison. For the two Xxx-to-Cys exchanges, | | | | | |
| # NOEs with side chain protons beyond βCH₂ were eliminated from the NOE upper distance constraints listed in the second column. Each of the disulfide bonds listed in the first column was enforced by the standard 3 upper and 3 lower distance constraints (Williamson et al., 1995). Each calculation was started with 100 randomized structures. | | | | | |

^{b}Number of NOE upper distance constraints in the input.
^{c}Residual DYANA target function value (Å²). The average ± standard deviation is given for a bundle of 20 conformers used to represent the structure.
^{d}Average number ± standard deviation of residual upper limit disulfied bond constraint violations larger than 0.1 Å. The number of residual lower limit disulfide bond constraint violations >0.1 Å was in all calculations between 0 and 1.
^{e}RMSD value (Å, average ± standard deviation) of the bundle of 20 conformers relative to the mean coordinates calculated for the backbone heavy atoms N, C^{α} and C′ of residues 125-228.
^{f}RMSD value (Å) between the mean structures of the mutant protein and hPrP(121-230) calculated for residues 125-228.

7. Stabilization of Globular Protein Structure at pH 7:

The globular protein stability of hPrP(121-230) and the variant proteins was measured monitoring the molar ellipticity at 222 nm in solutions containing different concentrations of guanidinium chloride (GdmCl).

FIG. 7 shows the GdmCl-dependent mean residue molar ellipticity of human prion proteins: (A) In buffer containing 20 mM sodium phosphate at pH 7.0 and (B) In buffer containing 20 mM sodium acetate at pH 5.0. The spectra in (A) and (B) were recorded with 30 &M protein solutions at 22° C.: filled squares, hPrP(121-230), open squares, hPrP(Ml66C/Y225C); filled circles, hPrP(M166C/E221C).

At pH 7.0, hPrP(121-230) undergoes a highly cooperative two-state transition (FIG. 7A) with a midpoint of transition [D]_{1/2}=2.1 M and a free energy of unfolding in the absence of denaturant ΔG⁰=−19 kJ mol⁻¹. These thermodynamic values are near identical to those determined for hPrP(90-231) (Swietnicki, W., Petersen, R., Gambetti, P. and Surewicz, W. K. (1997) pH-dependent stability and conformation of the recombinant human prion protein PrP(90-231). J Biol Chem, 272, 27517-27520). A single folding transition was also observed for the two hPrP(121-230) variant proteins (FIG. 7A). However, hPrP(M166C/E221C) and hPrP(M166C/Y225C) showed an increase in the transition midpoint [D_{1/2}], indicating that the global structures are stabilized by the engineered disulfide bonds (Fersht, A. R. (1993) The sixth Datta Lecture. Protein folding and stability: the pathway of folding of barnase. Febs Lett, 325, 5-16; Fersht, A. R. (1994) Jubilee Lecture. Pathway and stability of protein folding. Biochem Soc Trans, 22, 267-273). The lower folding cooperativity of the variant proteins indicates a deviation from a two-state folding mechanism.

8. Stabilization of α-Helix Versus β-Sheet Secondary Structure at pH 5:

At pH 5.0, hPrP(121-230) showed two distinctive folding transition regions with transition midpoints at 1.3 and 2.7 M GdmCl, clearly indicating the presence of a folding intermediate that is maximally populated at about 2 M GdmCl (FIG. 7B). The existence of a stable folding intermediate during equilibrium unfolding in GdmCl has also been reported for hPrP(90-231) and is observed when the pH of the buffer solution is less than of equal to 4.0 (Swietnicki et al., 1997). At pH 5.0, however, the unfolding curve of hPrP(90-231) was approximated by a two-state transition model. It thus seems that the flexibly disordered peptide segment 90-120 destabilizes the folding intermediate which accumulates at low GdmCl concentrations and acidic pH, presumably by interacting with the globular domain (Zahn et al., 2000).

Decreased solubility of the variant proteins precluded quantitative CD measurements below 1.2 M GdmCl concentration (FIG. 7B) so that we were not able to determine the folding transition model for these proteins. Similar to the data obtained at neutral pH, the observed folding transition midpoints of hPrP(M166C/E221C) and hPrP(M166C/Y225C) are shifted towards higher molarities of denaturant with respect to the second transition midpoint of hPrP(121-230), and the folding cooperativity is decreased (FIG. 7B).

To gain insight into the conformational properties of the human prion proteins under conditions corresponding to the presence of the stable folding intermediate of hPrP(121-230), we measured far-UV CD spectra at pH 5.0 in the presence and absence of 2 M GdmCl.

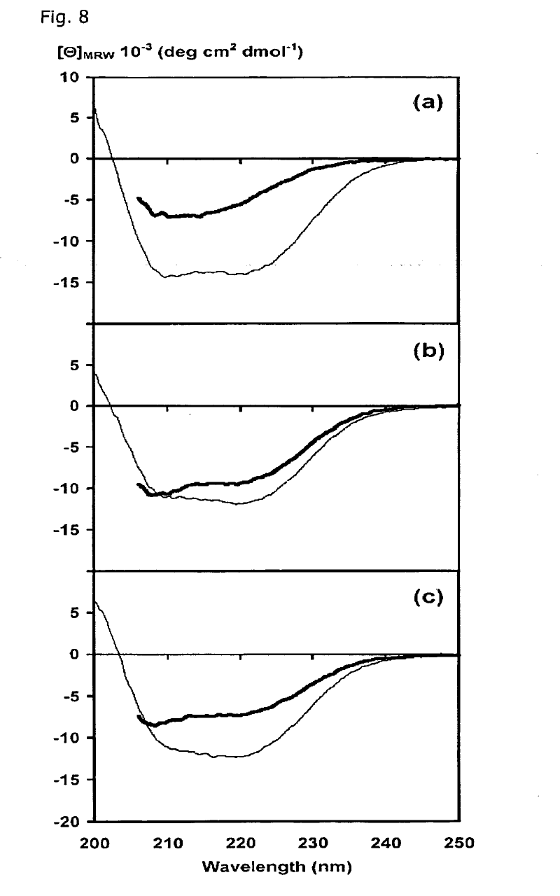

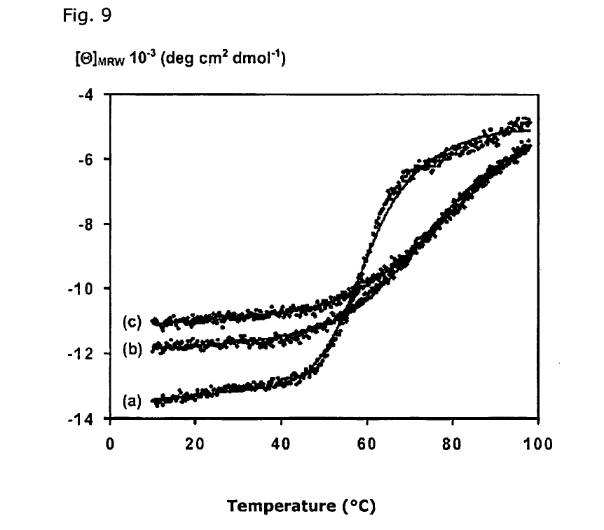

FIG. 8 shows circular dichroism spectra of human prion proteins: (A) hPrP(121-230), (B) hPrP(M166C/E221C), and (C) hPrP(M166C/Y225C). The spectra were recorded with 20 μM protein solutions in 20 mM sodium acetate at pH 5.0 and 22° C., either in the presence (bold line) or in the absence (thin line) of 2 M GdmCl.

The spectra of the three proteins in the absence of denaturant are essentially similar (FIG. 8), with the minima at 208 and 222 nm indicating a largely α-helical structure for all three proteins. The slight differences in the spectra of the variant proteins relative to the wild-type are presumably due to an additional absorption of the second disulfide-bond in the far-UV region (Coleman, D. L. and Blout, E. R. (1968) Optical activity of disulfide bond in L-cystine and some derivatives of L-cystine. J Am Chem Soc, 90, 2405-2416), since the structures are very similar apart from small localized changes around the disulfide bridge insertion points. Furthermore, it is known from NMR chemical shift measurements that the population of α-helical secondary structure within helix α3 of both hPrP(M166C/E221C) and hPrP(M166C/Y225C) is slightly decreased (FIG. 5).

At 2 M GdmCl, where the folding intermediate of hPrP(121-230) is maximally populated, the double minimum in the CD spectrum of hPrP(121-230) is replaced by a single minimum at 213 nm (FIG. 8A), which is characteristic of proteins rich in β-sheet secondary structure. A similar monomeric folding intermediate has been described at pH 4.0 for hPrP(90-231) which is maximally populated at 1 M GdmCl (Swietnicki et al., 1997), and also for mouse PrP(121-231) at 4 M urea (Hornemann, S. and Glockshuber, R. (1998) A scrapie-like unfolding intermediate of the prion protein domain PrP(121-231) induced by acidic pH. Proc Natl Acad Sci USA, 95, 6010-6014). It was postulated that these intermediates may represent a soluble precursor of PrP^{Sc}. Based on a more detailed insight into the mechanism of conformational transitions of hPrP(90-231) (Swietnicki, W., Morillas, M., Chen, S. G., Gambetti, P. and Surewicz, W. K. (2000) Aggregation and fibrillization of the recombinant human prion protein huPrP(90-231). Biochemistry-Us, 39, 424-431), it was found that the β-sheet intermediates oligomerize into large molecular weight aggregates that share some of the physical properties of PrP^{Sc} amyloid. These aggregates were not observed for mouse PrP(121-230) (Hornemann et al., 1998) and hPrP(121-231), presumably because these constructs are devoid of the peptide segment 90-120 that is required for the conformational transition of α-helix into β-sheet secondary structure during the PrP^{C} to PrP^{Sc} conversion in brain (Prusiner, S. B., Groth, D. F., Bolton, D. C., Kent, S. B. and Hood, L. E. (1984) Purification and structural studies of a major scrapie prion protein. Cell, 38, 127-134); Pan et al., 1993). Nonetheless, because of the similar CD spectra characteristic of β-sheet secondary structure we believe that all these intermediates are similar in nature and hence may be implicated in the conformatinal transition resulting in pathogenic protein.

The CD spectra of the two variant prion proteins in 2 M GdmCl are typical for a protein rich in α-helix structure (FIGS. 8B and 8C), indicating that there is no accumulation of a folding intermediate with increased β-sheet structure. The relative increase in amplitude at 208 nm versus 222 nm, when compared with the native protein, may be rationalized by a partial transition of α-helix into a random coil conformation, but there is no evidence for an α-helix-to-β-sheet transition in the presence of GdmCl, as is the case for wild-type protein.

FIG. 9 shows temperature-dependent mean residue molar ellipticity of human prion proteins: (A) hPrP(121-230), (B) hPrP(M166C/Y225C), and (C) hPrP(M166C/E221C). Protein concentration was 24 &M in 10 mM sodium acetate at pH 4.5.

The thermal unfolding of the three prion proteins under acidic conditions occurs in a single transition (FIG. 9), but the qualitatively different folding characteristics of wild-type versus variant proteins is also manifested in the thermal unfolding experiments. The two-state thermal unfolding of hPrP(121-230) is highly cooperative with a melting temperature of about 60° C. (FIG. 9A). The folding transition of hPrP(M166C/Y225C) and hPrP(M166C/E221C) is much less cooperative, and is shifted by more than 10° C. towards higher temperatures (FIGS. 9B and 9C), reflecting again the greater stability. The exact temperature of the folding transition could not be determined for the variant proteins, because even at 100° C. they have not reached the post-unfolding regime and contain a significant degree of protein secondary structure with negative ellipticity at 222 nm.

### DISCUSSION OF THE RESULTS

Comparison of the globular domains of human Dpl and human PrP reveals both close global similarities and marked local differences (T. Lührs, R. Riek, P. Güntert und K. Wüthrich, unpublished). Similar observations were reported for the corresponding murine proteins (Mo, H., Moore, R. C., Cohen, F. E., Westaway, D., Prusiner, S. B., Wright, P. E. and Dyson, H. J. (2001) Two different neurodegenerative diseases caused by proteins with similar structures. Proc Natl Acad Sci USA, 98, 2352-2357). Within the region of the hDpl structure that corresponds to the presumed factor X epitope in hPrP (Telling et al., 1994; Prusiner, 1998), the helix α3 is shortened by more than two turns, and the C-terminal peptide segment 144-149 is folded against the loop connecting α2 and α2. Between the two proteins, the plane of the β-sheet, which immediately precedes the factor X epitope in the PrP sequence, is rotated by about 180° with respect to the molecular scaffold formed by the helical secondary structures, and in hDpl it is located two residues closer to the helix α1. Furthermore, in hDpl the helix α2 of hPrP, which follows the factor X epitope in the PrP sequence, is replaced by two shorter helices α2^{a} and α2^{b}. The present structure determination of a mutant human prion protein containing two disulfide bonds now enables novel insights into the relations between the molecular structures of hPrP and hdpl in the region of the factor X epitope, which may also support the ongoing search for the still unknown functions of the two proteins.

The global structure of the mutant hPrP is similar to both wild-type hPrP and hDpl. The RMSD value between the backbone heavy atoms of residues involved in the common α-helices of the mean structures of hPrP(M166C/E221C) and hDpl is 1.69 Å. After superposition of the three-dimensional structures for minimal RMSD of this scaffold, the positions and orientations of the artificial disulfide bond in hPrP(M166C/E221C) and of the corresponding natural disulfide bond in hDpl are closely similar. This is rather surprising, considering that in hDpl the disulfide bond Cys94-Cys145 connects two segments without regular secondary structure, i.e., the loop 91-100 and the C-terminal segment of residues 142-153, whereas in the mutant hPrP the disulfide Cys166-Cys221 anchors the mobile loop 165-172 against the helix α3 (see FIG. 2). From the present data it appears plausible that the local structure of the presumed factor X epitope observed in PrP might initially also have been present in Dpl, with a cysteinyl residue at position 94 forming a disulfide bond with a second cysteinyl located in a position that would have been compatible with the helix α3 extending all the way to the C-terminus, e.g., position 147 (FIG. 2). During further evolution, a deletion in helix α3 could have relocated this cysteinyl into position 145 (FIG. 1), where it is incompatible with regular α-helix structure beyond about residue 141. Nature would then appear to have selected for this disulfide bridge and the poorly structured C-terminal peptide segment. Since this disulfide bond is common to all known mammalian Dpl sequences (Moore et al., 1999), there is a clear indication mammalian Dpl sequences (Moore et al., 1999), there is a clear indication that this intriguing choice of the Cys position nearest to the C-terminus has a specific role in the physiological Dpl function. A different function of this structural region of Dpl from that of the factor X epitope in PrP is independently suggested by the fact that the loop 91-100 contains an Asn-linked glycosylation site at position 98 (Moore et al., 1999), which has no counterpart in PrP. If factor X interactions are indeed essential for prion propagation, then the disulfide-related different conformation in this molecular region of Dpl may provide a rationale for the observation that no evidence could so far be obtained for a TSE that would be caused by Dpl (Behrens, A., Brandner, S., Genoud, N., and Aguzzi, A. (2001) Normal neurogenesis and scrapie pathogenesis in neural grafts lacking the prion protein homologue Doppel. EMBO Rep, 2, 347-352; Tuzi, N. L., Gall, E., Melton, D. and Manson, J. C. (2002) Expression of doppel in the CNS of mice does not modulate transmissible spongiform encephalopathy disease. J Gen Virol, 83, 705-711).

Currently there are no drugs available for the treatment of prion diseases in humans and animals. Within the framework of the protein-only hypothesis (Prusiner, 1998), at least two mechanisms can be imagined that could prevent the accumulation of toxic protein conformations resulting in TSE. The first mechanism relies on a “PrP^{C}-binder” that specifically binds to the normal form of the prion protein, thus preventing PrP^{C} from folding into PrP^{Sc}. In the context of the “nucleated polymerisation” model (Jarrett, J. T. and Lansbury, P. T., Jr. (1993) Seeding “one-dimensional crystallization” of amyloid: a pathogenic mechanism in Alzheimer's disease and scrapie? Cell, 73, 1055-1058), proposing that PrP^{C} and PrP^{Sc} are in a rapidly established equilibrium, PrP^{C}-binding molecules may simply stabilize the native protein conformation of PrP^{C} thus slowing down conversion kinetics by decreasing the concentration of polymerisation nuclei. In the context of the “template-assisted” model (Prusiner, S. B., Scott, M., Foster, D., Pan, K. M., Groth, D., Mirenda, C., Torchia, M., Yang, S. L., Serban, D., Carlson, G. A. and et al. (1990) Transgenetic studies implicate interactions between homologous PrP isoforms in scrapie prion replication. Cell, 63, 673-686), PrP^{C}-binders may directly block the binding site of PrP^{Sc} or another molecule that is required for prion propagation, such as protein X (Telling et al., 1994; 1995). The second mechanism would use a “PrP^{Sc}-binder” to either block the homophilic assembly of PrP^{Sc} into amyloid fibrils or to interfere with a heterophilic interaction with other macromolecules that otherwise are implicated in pathogenic pathways. The advantage of PrP^{Sc}-binders over PrP^{C}-binders is that they do not interfere with the yet unknown physiological function of the cellular form of the protein.

Several recent reports indicate that antibodies directed against PrP^{C} have the potential to eliminate the transmissible agent of spongiform encephalopathies from scrapie-infected cells in vitro (Enari, M., Flechsig, E. and Weissmann, C. (2001) Scrapie prion protein accumulation by scrapie-infected neuroblastoma cells abrogated by exposure to a prion protein antibody. Proc Natl Acad Sci USA, 98, 9295-9299; Horiuchi, M. and Caughey, B. (1999) Specific binding of normal prion protein to the scrapie form via a localized domain initiates its conversion to the protease-resistant state. Embo J, 18, 3193-3203). A humoral immune response could prevent scrapie pathogenesis in vivo, indicating that induction of protective anti-prion immunity appears to be feasible (Heppner, F. L., Musahl, C., Arrighi, I., Klein, M. A., Rulicke, T., Oesch, B., Zinkernagel, R. M., Kalinke, U. and Aguzzi, A. (2001) Prevention of scrapie pathogenesis by transgenic expression of anti-prion protein antibodies. Science, 294, 178-182). Most of these studies identified the region encompassing residues 132-156 of the prion protein as the target for antibody binding (Heppner et al., 2001). Various chemical compounds have been described that bind to PrP^{C} and inhibit prion propagation in ScN2a cells, but only a few show transient therapeutic effect in animal experiments (Gilch, S., Winklhofer, K. F., Groschup, M. H., Nunziante, M., Lucassen, R., Spielhaupter, C., Muranyi, W., Riesner, D., Tatzelt, J. and Schätzl, H. M. (2001) Intracellular re-routing of prion protein prevents propagation of PrP^{Sc} and delays onset of prion disease. Embo J, 20, 3957-3966). The antimalarial drug quinacrine has recently been identified as a promising lead compound for the treatment of Creutzfeldt-Jakob disease (CJD) (Doh-Ura, K., Iwaki, T. and Caughey, B. (2000) Lysosomotropic agents and cysteine protease inhibitors inhibit scrapie-associated prion protein accumulation. J Virol, 74, 4894-4897; Korth, C., May, B. C. H., Cohen, F. E. and Prusiner, S. B. (2001) Acridine and phenothiazine derivatives as pharmacotherapeutics for prion disease. Proc Natl Acad Sci USA, 98, 9836-9841). The quinacrine binding site of recombinant human PrP has been mapped to residues Tyr225, Tyr226, and Gln227 of helix α3, which is located near the “protein X” epitope (Vogtherr, M., Grimme, S., Elshorst, B., Jacobs, D. M., Fiebig, K., Griesinger, C. and Zahn, R. (2003) Antimalarial drug quinacrine binds to C-terminal helix of cellular prion protein. J Med Chem, (in press)). The millimolar dissociation constant of the quinacrine-PrP^{C} complex suggests that this drug inhibits prion propagation within endolysosomes, where it is 10,000-fold concentrated (O'Neill, P. M., Bray, P. G., Hawley, S. R., Ward, S. A. and Park, B. K. (1998) 4-aminoquinolines—Past, present, and future: A chemical perspective. Pharmacol Ther, 77, 29-58). Although recovery of patients treated with quinacrine was only transient (Follette, P. (2003) New perspectives for prion therapeutics meeting: Prion disease treatment's early promise unravels. Science, 299, 191-192), second generation drugs based on quinacrine and analogues thereof hold the promise of more potent drugs for the treatment of CJD (May, B. C. H., Fafarman, A. T., Hong, S. B., Rogers, M., Deady, L. W., Prusiner, S. B. and Cohen, F. E. (2003) Potent inhibition of scrapie prion replication in cultured cells by bis-acridines. Proc Natl Acad Sci USA, 100, 3416-3421).

Only a few compounds with therapeutic potential have been identified which specifically bind to the scrapie conformation of prion proteins. A monoclonal PrP^{Sc}-binding antibody was discovered in 1997 (Korth, C., Stierli, B., Streit, P., Moser, M., Schaller, O., Fischer, R., SchulzSchaeffer, W., Kretzschmar, H., Raeber, A., Braun, U., Ehrensperger, F., Hornemann, S., Glockshuber, R., Riek, R., Billeter, M., Wüthrich, K. and Oesch, B. (1997) Prion (PrP^{Sc})-specific epitope defined by a monoclonal antibody. Nature, 390, 74-77), but there are no recent reports confirming the specificity of binding activity in vivo. Soto and coworkers constructed a 13-residue β-sheet breaker peptide that partially reverses in vitro PrP^{Sc} to a PrP^{C}-like protein (Soto, C., Kascsak, R. J., Saborio, G. P., Aucouturier, P., Wisniewski, T., Prelli, F., Kascsak, R., Mendez, E., Harris, D. A., Ironside, J., Tagliavini, F., Carp, R. I. and Frangione, B. (2000) Reversion of prion protein conformational changes by synthetic β-sheet breaker peptides. Lancet, 355, 192-197). The peptide was also active in intact cells and delayed the appearance of clinical symptoms in mice with experimental scrapie.

Another strategy that has been suggested for TSE treatment is based on the design of soluble PrP derivatives that bind to PrP^{Sc}, but cannot be converted by a template-assisted mechanism and thus inactivate the bound PrP^{Sc} molecule. Bürkle and coworkers have shown that the presence of amino acids 114-121 of mouse PrP plays an important role in the conversion of PrP^{C} into PrP^{Sc} and that a deletion mutant lacking these residues behaves as a dominant negative mutant with respect to PrP^{Sc} accumulation in cell culture (Hölscher, C., Delius, H. and Bürkle, A. (1998) Overexpression of nonconvertible PrP^{C} D114-121 in scrapie-infected mouse neuroblastoma cells leads to trans-dominant inhibition of wild-type PrP^{Sc} accumulation. J Virol, 72, 1153-1159). Dominant negative inhibition could thus form a basis for treatment or prevention of prion diseases. Recently, Aguzzi and coworkers constructed a soluble dimeric prion protein that binds PrP^{Sc} in vivo and antagonizes prion disease (Meier, P., Genoud, N., Prinz, M., Maissen, M., Rulicke, T., Zurbriggen, A., Raeber, A. J. and Aguzzi, A. (2003) Soluble dimeric prion protein binds PrP^{Sc} in vivo and antagonizes prion disease. Cell, 113, 49-60). The soluble dimeric PrP consisted of full-length murine PrP fused to the Fcγ-tail of human IgG₁. Following intracerebral or intraperitoneal innoculation with prion, substochiometric amounts of this soluble protein significantly delayed disease onset in Prnp^{+/+} mice. These studies provide evidence that soluble PrP fusion proteins may be used for prion disease therapeutics.

The combination of structural and thermodynamical data on hPrP(M166C/E221C) and hPrP(M166C/Y225C) suggests that disulfide variants of PrP may also be applicable as a dominant negative treatment for prion diseases. The preservation of the native three-dimensional structure in the PrP variants makes it likely that these have a similar affinity to PrP^{Sc} as does wild-type PrP^{C}. The PrP^{Sc} binding site of PrP^{C} is not known, but there is evidence from genetic experiments (Telling et al., 1994; 1995) that the region of helix α1 (residues 144-154 in human PrP) and the preceding loop region (residues 132-143 in human PrP) are involved in PrP^{Sc} binding. This region is structurally unchanged after introduction of an additional disulfide bond between helix α3 and the loop connecting helix α2 and the second β-strand (FIG. 4). In the context of the “template-assisted” PrP model for prion propagation (Prusiner, 1998), the increased [D]_{1/2}-values (FIG. 7) and melting temperatures (FIG. 9) of the variant proteins indicate that a higher amount of free energy is required for transforming PrP^{Sc}-bound PrP^{C} into a conformation that is competent for folding into PrP^{Sc}. Therefore, the additional disulfide bonds in the variant proteins should per se decrease efficiency of prion propagation and thus delay progression of prion disease. At pH values between 4.7 and 5.8 in endosomes (Lee, R. J., Wang, S. and Low, P. S. (1996) Measurement of endosome pH following folate receptor-mediated endocytosis. Biochim Biophys Acta, 1312, 237-242) the protection against a conversion into a pathogenic protein conformation presumably is even more pronounced, because the α-helix secondary structure of the variant proteins resists a conformational transition into β-sheet secondary structure (FIGS. 8B and 8C). Thus, within the environment of endosomes or lysosomes PrP^{Sc} would probably become trapped in a complex with a bound PrP^{C} disulfide variant.

It will be of interest, to investigate if such a mechanism can be established during cell culture and animal experiments, where the recombinant disulfide variant prion proteins are intracerebraly or intraperitonealy innoculated or expressed in vivo using a gene therapeutic approach. If successful, introduction of stabilizing disulfide bonds into PrP^{C} might be employed for the treatment of a variety of neurodegenerative diseases.

### MATERIALS AND METHODS

1. Sample Preparation and Characterization:

For the cloning, expression and purification of the two-disulfide hPrP(121-230) mutants in unlabeled form and with uniform ¹³C,¹⁵N-labeling we closely followed the strategy used for the preparation of wild-type hPrP (Zahn et al., 1997, 2000), where double-residue exchanges were constructed following the Quickchange site-directed mutagenesis protocol (Stratagene). 1 mM protein solutions in 90% H₂O/10% D₂O containing 10 mM [d₄]-sodium acetate at pH 4.5 for NMR spectroscopy were obtained using Ultrafree-15 Centrifugal Filter Biomax Devices (Millipore).

2. NMR Measurements and Structure Calculations:

The NMR measurements were performed on Bruker DRX500, DRX600 and DRX750 spectrometers equipped with four radio-frequency channels and triple resonance probeheads with shielded z-gradient coils. For the collection of conformational constraints, a three-dimensional combined ¹⁵N/¹³C-resolved [¹H,¹H]-NOESY spectrum (Boelens, R., Burgering, M., Fogh, R. H. and Kaptein, R. (1994) Time-saving methods for heteronuclear multidimensional NMR of (¹³C, ¹⁵N) doubly labeled proteins. J. Biomol. NMR 4, 201-213) in H₂O was recorded with a mixing time Tₘ=40 ms at T=20° C., 256(t₁)×50(t₂)×1024(t₃) complex points, t_{1,max}(¹H)=28.4 ms, t_{2,max}(¹⁵N)=20.6 ms, t_{2,max}(¹³C)=8.3 ms, and t_{3,max}(¹H)=97.5 ms, and this data set was zero-filled to 512×128×2048 points. Processing of the spectra was performed with the program PROSA (Güntert, P., Dötsch, V., Wider, G. and Wüthrich, K. (1992) Processing of multidimensional NMR data with the new software PROSA. J. Biomol. NMR 2, 619-629). The ¹H, ¹⁵N and ¹³C chemical shifts have been calibrated relative to 2,2-dimethyl-2-silapentane-5-sulfonate, sodium salt.

Steady-state ¹⁵N{¹H}-NOEs were measured at 600 MHz following Farrow, N. A., Zhang, O., Forman-Kay, J. D. and Kay, L. E. (1994) A heteronuclear correlation experiment for simultaneous determination of 15N longitudinal decay and chemical exchange rates of systems in slow equilibrium. J. Biomol. NMR 4, 727-734), using a proton saturation period of 5 s by applying a cascade of 120-degree pulses in 20 ms intervals; t_{1,max}(¹⁵N)=61.0 ms, t_{2,max}(¹H)=142.6 ms, time domain data size 152×1024 complex points.

NOE assignment was obtained using the program CANDID (Herrmann et al., 2002) in combination with the structure calculation program DYANA (Güntert et al., 1997). CANDID and DYANA perform automated NOE-assignment and distance calibration of NOE intensities, removal of covalently fixed distance constraints, structure calculation with torsion angle dynamics, and automatic NOE upper distance limit violation analysis. As input for CANDID, peak lists of the aforementioned NOESY spectrum were generated by interactive peak picking with the program XEASY (Bartels, C., Xia, T. H., Billeter, M., Güntert, P. and Wüthrich, K. (1995) The program XEASY for computer-supported NMR spectral analysis of biological macromolecules. J. Biomol. NMR 6, 1-10) and automatic integration of the peak volumes with the program SPSCAN (Ralf Glaser, personal communication). The input for the calculations with CANDID and DYANA contained these peak lists, a chemical shift list from the previous sequence-specific resonance assignment, and dihedral angle constraints for the backbone angles φ and ψ that were derived from ¹³C^{α} shifts (Luginbühl et al., 1995). The calculation followed the standard protocol of 7 cycles of iterative NOE assignment and structure calculation (Herrmann et al., 2002). During the first six CANDID cycles, ambiguous distance constraints were used. For the final structure calculation, only NOE distance constraints were retained that correspond to NOE cross peaks with unambiguous assignment after the sixth cycle of calculation. Stereospecific assignments were identified by comparison of upper distance limits with the structure resulting from the sixth CANDID cycle. The 20 conformers with the lowest final DYANA target function values were energy-minimized in a water shell with the program OPALp (Luginbühl, P., Güntert, P., Billeter, M. and Wüthrich, K. (1996) The new program OPAL for molecular dynamics simulations and energy refinements of biological macromolecules. J. Biomol. NMR 8, 136-146), using the AMBER force field (Cornell, W. D., Cieplak, P., Bayly, C. I., Gould, I. R., Merz, K. M., Jr., Ferguson, D. M., Spellmeyer, D. C., Fox, T., Caldwell, J. W. and Kollman, P. A. (1995) A second generation force field for the simulation of proteins, nucleic acids, and organic molecules. J. Am. Chem. Soc. 117, 5179-5197). The program MOLMOL (Koradi, R., Billeter, M. and Wüthrich, K. (1996) MOLMOL: A program for display and analysis of macromolecular structures. J. Molec. Graphics 14, 51-55) was used to analyze the resulting 20 energy-minimized conformers (Tables I and II) and to prepare drawings of the structures.

3. CD Measurements and Equilibrium Experiments:

Circular dichroism spectra were recorded with a Jasco J720 spectropolarimeter interfaced with a Peltier-type temperature control unit, with 1 mm or 0.2 mm pathlength cuvette. The ellipticity at 222 nm was used for monitoring GdmCl-induced unfolding, and denaturation curves were fitted to a two-state model, assuming that the free energy ΔG of unfolding is lineary dependent of the concentration of denaturant [D] present in the solution (Bolen, D. W. and Santoro, M. M. (1988) Unfolding free-energy changes determined by the linear extrapolation method. 2. Incorporation of delta G degrees N-U values in a thermodynamic cycle. Biochemistry-Us, 27, 8069-8074; Santoro, M. M. and Bolen, D. W. (1988) Unfolding free-energy changes determined by the linear extrapolation method. 1. Unfolding of phenylmethanesulfonyl alpha-chymotrypsin using different denaturants. Biochemistry-Us, 27, 8063-8068): ΔG=ΔG⁰+m[D], where m ist the cooperativity of unfolding and ΔG⁰ is ΔG in the absence of denaturant. Evaluation of the equilibrium constants in the transition region was obtained by extrapolation of the pre- and post-transitional baselines into the transition region. The two-state model used to fit the data assumes the following dependence of the observed signal S^{obs}: S_{obs}=((S_{N}+m_{N}[D])exp(−(ΔG⁰+m[D])/RT)+S_{D}+m_{D}[D])/(1+exp(−(ΔG⁰+m[D])/RT)), where S_{N} and S_{D} are the intercepts and m_{N} and m_{D} are the slopes of the pre- and post-transition regimes, respectively, T is the absolute temperature in Kelvin and R is the gas constant. Thus, at the transition midpoint [D_{1/2}], where the argument in the exponentials must vanish: [D]_{1/2}=ΔG⁰/m.

Thermal denaturation experiments were performed monitoring the circular dichroism at 222 nm, while changing the temperature from 10° C. to 90° C. with a constant rate of change of 50° C. per hour.

1. A mutant of a protein, the protein causing a disease after having performed a conformational transition, the disease comprising:
a) neurodegenerative diseases of the group comprising Transmissible Songiform Encephalopathy (TSE), Alzheimers disease, Multiple Sclerosis, and Parkinsons disease; and/or other
b) conformational diseases of the group comprising Primary systematic amyloidosis, Type II diabetes, and Atrial amyloidosis;
wherein the mutant protein or a variant of which comprises at least one additional engineered disulfide bond which inhibits a conformational transition of such proteins in human and animals.

2. The protein of claim 1,
wherein the at least one additional disulfide bond is engineered at a position similar to a disulfide bond in a structurally related non-pathogenic protein.

3. The protein of claim 1,
wherein the protein is a prion protein, the at least one engineered additional disulfide bond being situated in the gobular domain.

4. The prion protein of claim 3,
wherein the at least one engineered additional disulfide bond is situated in a position similar as in the doppel protein (Dpl).

5. The prion protein of claim 3,
wherein the protein comprises a ‘factor X’ binding epitope and the at least one engineered additional disulfide bond is situated within this ‘factor X’ binding epitope.

6. The prion protein of claim 3,
wherein the at least one engineered additional disulfide bond is introduced between a first segment comprising the amino acid residues 165-175 and a second segment comprising the C-terminal amino acid residues 215-230 in a human prion protein or between structurally corresponding amino acid segments in other species.

7. The prion protein of claim 6,
wherein the at least one engineered additional disulfide bond is linking amino acid residues M166C and E221C or amino acid residues M166C and Y225C.

8. A nucleic acid sequence coding for a mutant protein, the protein causing a disease after having performed a conformational transition, the disease comprising:
a) neurodegenerative diseases of the group comprising Transmissible Spongiform Encephalopathy (TSE), Alzheimers disease, Multiple Sclerosis, and Parkinsons disease; and/or other
b) conformational diseases of the group comprising Primary systematic amyloidosis, Type II diabetes, and Atrial amyloidosis;
wherein the nucleic acid sequence is coding for the mutant protein or a variant of which that comprises at least one additional engineered disulfide bond which inhibits a conformational transition of such proteins in human and animals.

9. Plasmid constructs, vectors, transformed cells, transgenic animals including cattle, sheep, cat, elk, and deer, and recombinant proteins, comprising and/or being encoded by a nucleic acid sequence according to claim 8.

10. Use of a mutant of a protein, the protein causing a disease after having performed a conformational transition, the disease comprising:
a) neurodegenerative diseases of the group comprising Transmissible Spongiform Encephalopathy (TSE), Alzheimers disease, Multiple Sclerosis, and Parkinsons disease; and/or other
b) conformational diseases of the group comprising Primary systematic amyloidosisi, Type II diabetes, and Atrial amyloidosis;
the mutant protein or a variant of which comprising at least one additional engineered disulfide bond which inhibits a conformational transition of such proteins in human and/or animals, wherein the mutant protein is used for therapeutic treatment of conformational diseases.

11. Use of a mutant of a protein, the protein causing a disease after having performed a conformational transition, the disease comprising:
a) neurodegenerative diseases of the group comprising Transmissible Spongiform Encephalopathy (TSE), Alzheimers disease, Multiple Sclerosis, and Parkinsons disease; and/or other
b) conformational diseases of the group comprising Primary systematic amyloidosisi, Type II diabetes, and Atrial amyloidosis;
the mutant protein or a variant of which comprising at least one additional engineered disulfide bond which inhibits a conformational transition of such proteins in human and/or animals, wherein the mutant protein is used for the manufacturing of a medicament for the therapeutic treatment of conformational diseases.

12. The use according to claim 10,
wherein the engineered additional disulfide bond(s) prevent(s) a conformational transition of mutant PrP^{C} into PrP^{Sc} and thus suppress(es) a conformational transition of mutant PrP^{C} into PrP^{Sc} in co-existing wild-type proteins by dominant negative inhibition.

13. The use according to claim 10,
wherein the conformational transition of wild type PrP^{C} into PrP^{Sc} oligomers is suppressed by binding of mutant PrP^{C} to wild-type PrP^{C}.

14. The use according to claim 10,
wherein the conformational transition of wild type PrP^{Sc} oligomers into PrP^{Sc} amyloid fibrils is suppressed by binding of mutant PrP^{C} to wild-type PrP^{Sc} oligomers.

15. The use according to claim 10,
wherein the conformational transition of wild type PrP^{C} into PrP^{C}/PrP^{Sc} heterodimers is suppressed by binding of mutant PrP^{C} to wild-type PrP^{Sc}.

16. The use according to claim 10,
wherein the elongation of amyloid fibrils or the dissociation of amyloid fibrils into PrP^{Sc} oligomers is suppressed by binding of mutant PrP^{C} to wild-type PrP^{Sc} amyloid fibrils.

17. The use according to claim 8,
wherein in vivo generation of disulfide mutants of prion proteins or variants thereof is carried out in order to enable an intended therapy of Transmissible Spongiform Encephalopathy (TSE) in human, e.g. by somatic gene therapy with lentiviral vector, where TSE includes spontaneous, in herited, iatrogenic and variant forms of Creutzfeldt-Jakob disease (CJD), fatal familial insomnia (FFI), and Gerstmann-Sträussler-Scheinker syndrome (GSS).

18. The use according to claim 10,
wherein the recombinant production of disulfide mutants of prion proteins or variants thereof is carried out in order to enable an intended therapy of TSE in human, e.g. by direct application of the recombinant protein, where TSE includes spontaneous, inherited, iatrogenic and variant forms of CJD, FFI, and GSS.

19. The use according to claim 8,
wherein in vivo generation of disulfide mutants of prion proteins or variants thereof is carried out in order to enable an intended therapy of TSE in animals, e.g. by somatic gene therapy with lentiviral vector, where TSE includes bovine spongiform encephalopathy (BSE), scrapie in sheep, feline spongiform encephalopathy (FSE), and chronic wasting disease (CWD) in elk and deer.

20. The use according to claim 10,
wherein recombinant production of disulfide mutants of prion proteins or variants thereof is carried out in order to enable an intended therapy of TSE in animals, e.g. by direct application of the recombinant protein, where TSE includes BSE, scrapie, FSE and CWD.

21. The use according to claim 10,
wherein recombinant production of disulfide mutants of prion proteins or variants thereof is carried out as “conversion-resistant PrP^{C} standard” for TSE-tests applied to human or animals, where recombinant PrP^{C} is amplified by PrP^{Sc} from pathogenic tissue or bodily fluid such as blood and urine.

22. The use according to claim 10,
wherein in vivo generation of disulfide mutants of prion proteins or variants thereof is carried out in order to enable breeding of TSE-resistant animals by somatic gene therapy with lentiviral vector, where animals include cattle, sheep, cat, elk, deer, pig, horse, and fish.

23. Medicament for the treatment of:
a) neurodegenerative diseases of the group comprising Transmissible Spongiform Encephalopathy (TSE), Alzheimers disease, Multiple Sclerosis, and Parkinsons disease; and/or other
b) conformational diseases of the group comprising Primary systematic amyloidosis, Type II diabetes, and Atrial amyloidosis;
in humans, the medicament comprising a mutant protein or a variant of which comprising at least one additional engineered disulfide bond which inhibits a conformational transition of such proteins.

24. Medicament for the treatment of bovine spongiform encephalopathy (BSE), scrapie in sheep, feline spongiform encephalopathy (FSE), and chronic wasting disease (CWD) in elk and deer, the medicament comprising a mutant protein or a variant of which comprising at least one additional engineered disulfide bond which inhibits a conformational transition of such proteins.
